(19) United States
(12) Patent Application Publication
(10) Pub. No.: US 2011/0196219 A1
(43) Pub. Date: Aug. 11, 2011

(54) Fetal Scalp Blood Analyzer

(76) Inventor: Kathleen Edmunds, Knoxville, TN (US)

(21) Appl. No.: 12/701,654

(22) Filed: Feb. 8, 2010

(51) Int. Cl. A61B 5/151; A61B 5/00

(52) U.S. Cl. 600/368; 600/583

(57) ABSTRACT

A hand-held analyzer for collecting a sample of blood from the scalp of a fetus and measuring the pH of the blood sample. The analyzer includes a probe with a blunt proximal end that facilitates a firm and positive placement of the probe against the scalp. The probe includes a capillary tube for collecting blood and an extensible lancet which at rest is positioned within the capillary tube. During operation, the lancet is extended to make an incision in the scalp. The lancet then retreats, and blood flows into the capillary tube. Sensors for measuring pH are in liquid communication with the capillary tube. A display on or near the hand-held analyzer shows a readout of the fetal scalp blood pH. Measuring the pH of the fetal scalp blood in utero can provide early detection of acidosis and likely hypoxia.

### CROSS-REFERENCE TO RELATED APPLICATIONS

Not Applicable

### STATEMENT REGARDING FEDERALLY-SPONSORED RESEARCH OR DEVELOPMENT

Not Applicable

### BACKGROUND OF THE INVENTION

1. Field of Invention

The present invention relates generally to a medical diagnostic testing device. More particularly, the invention is directed toward a diagnostic testing device in which a liquid collector is combined with a sharp edge or point for piercing skin to draw blood and test the pH of the blood.

2. Description of the Related Art

Fetal hypoxia-ischemia is believed to be a leading cause of cerebral palsy. Studies have indicated a strong correlation between hypoxia and acidosis. Measuring the pH of the scalp in utero can provide early detection of acidosis and likely hypoxia. The preferred detection method involves sampling fetal scalp blood and measuring the pH of that blood sample. General practice for the procedure involves inserting a plastic cone into the vagina after the expecting mother's cervix is dilated at 3 cm or more. The fetus's scalp is cleaned with a cotton swab, a small laceration is made in the scalp, and blood droplets are collected in a capillary tube. The blood sample is then tested with a blood gas analyzer—usually in the hospital laboratory, but sometimes in the delivery room itself. This traditional method requires that the expecting mother's cervix is dilated at 3 cm or more. The traditional method also involves a delay, often of several minutes, between the laceration and reporting a blood pH result to medical personnel.

Other devices have been developed to address these and other problems. Typical of the art are those devices disclosed in the following U.S. patents: U.S. Pat. No. 4,320,764, issued Mar. 23, 1982 to Hon; U.S. Pat. No. 4,360,016, issued Nov. 23, 1982 to Sarrine; U.S. Pat. No. 4,441,510, issued Apr. 10, 1984 to Worley, deceased et al.; U.S. Pat. No. 4,658,825, issued Apr. 21, 1987 to Hochberg et al.; U.S. Pat. No. 4,660,570, issued Apr. 28, 1987 to Dombrowski; U.S. Pat. No. 5,551,424, issued Sep. 3, 2996 to Morrison et al.; U.S. Pat. No. 6,058,321, issued May 2, 2002 to Swayze et al.; and U.S. Pat. No. 6,423,011, issued Jul. 23, 2002 to Arulkulmaran et al. Also typical of the art is the device disclosed in U.S. Patent Publication No. 2004/0092843 A1, published on May 12, 2004, by Kreiser et al.

### BRIEF SUMMARY OF THE INVENTION

A hand-held analyzer for collecting a sample of blood from the scalp of a fetus and measuring the pH of the blood sample is disclosed. The analyzer includes a probe with a blunt proximal end that facilitates a firm and positive placement of the probe against the scalp. The probe includes a capillary tube for collecting blood and an extensible lancet which at rest is positioned within the capillary tube. During use of the analyzer, the lancet is extended to make an incision in the scalp. The lancet then retreats, and blood flows into the capillary tube. Sensors for measuring pH are in liquid communication with the interior of the capillary tube. A display on or near the hand-held analyzer shows the fetal blood pH.

### BRIEF DESCRIPTION OF THE DRAWINGS

The above-mentioned features of the invention will become more clearly understood from the following detailed description of the invention read together with the drawings in which:

FIG. 1 is a block diagram illustrating one embodiment of the invention;

FIG. 2 is a perspective view of one embodiment of the invention;

FIG. 3 is a side-on view of the embodiment shown in FIG. 2; and

FIG. 4 is a view of the probe portion the embodiment shown in FIGS. 2 and 3.

### DETAILED DESCRIPTION OF THE INVENTION

The present invention provides a fetal scalp blood analyzer that combines the functions of lancing the scalp, collecting a blood sample, and analyzing the blood sample to measure the pH of the fetal scalp blood. The analyzer includes a probe with a capillary tube and an extensible lancet positioned within the capillary tube. The analyzer further includes at least one pH sensor in liquid communication with the interior of the capillary tube, a controller adapted to receive information from the sensor and measure blood pH, and a display adapted to report the blood pH.

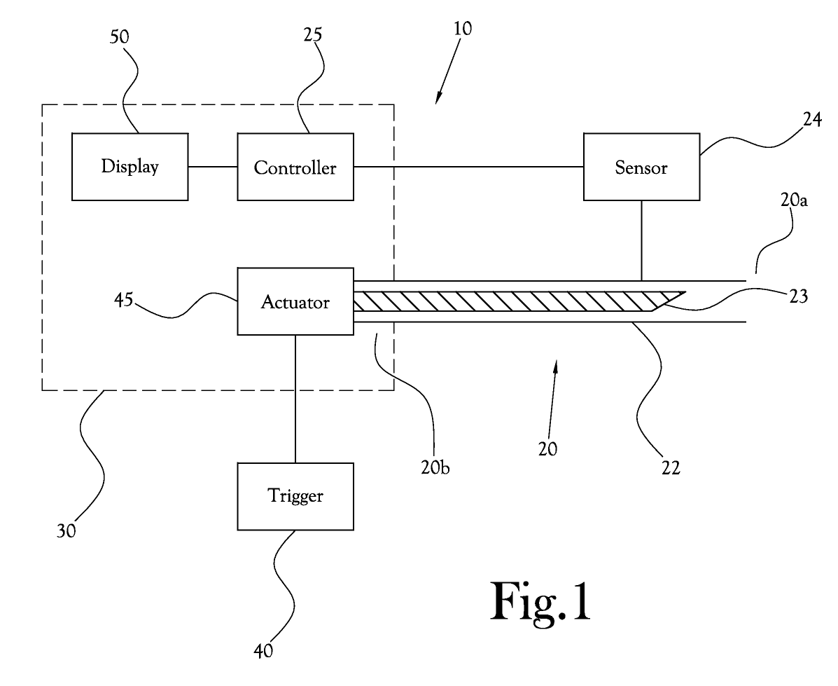

FIG. 1 illustrates one embodiment of the present invention. The analyzer 10 comprises a probe 20 and a housing 30. The probe 20 has a proximal end 20a and a distal end 20b, with the proximal end 20a defining an aperture. The probe 20 includes a capillary tube 22 and a lancet 23 positioned within the capillary tube 22. The lancet 23 is adapted to extend out of and retreat into the capillary tube through the aperture in the proximal end 20a of the probe 20. A sensor 24 adapted to measure pH is in liquid communication with the interior of the capillary tube 22. The housing 30 includes a controller 25 that receives information from the sensor 24 and a display 50 that shows an operator the pH of the drawn blood. A trigger 40 is connected to an actuator 45 that controls the extension and retraction of the lancet 23.

During use, an operator inserts the probe 20 into the birth canal until the proximal end 20a of the probe 20 has made contact with the scalp of the fetus. Next, the operator uses the trigger 40 to signal the actuator 45 to extend the lancet 23. The lancet 23 makes a small incision in the scalp sufficient to produce blood. The lancet 23 then retreats within the capillary tube 22. Blood flows into the capillary tube 22, where the sensor 24 measures the pH of the blood. The sensor 24 communicates its information to the controller 25, which in turn communicates with the display 50 to show the operator the pH of the drawn blood.

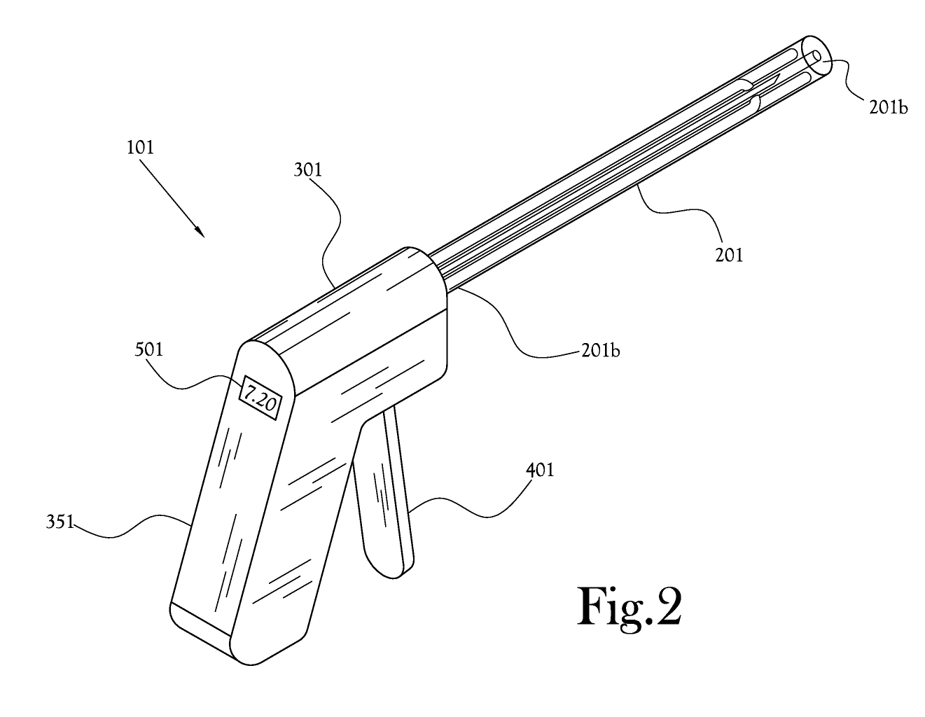

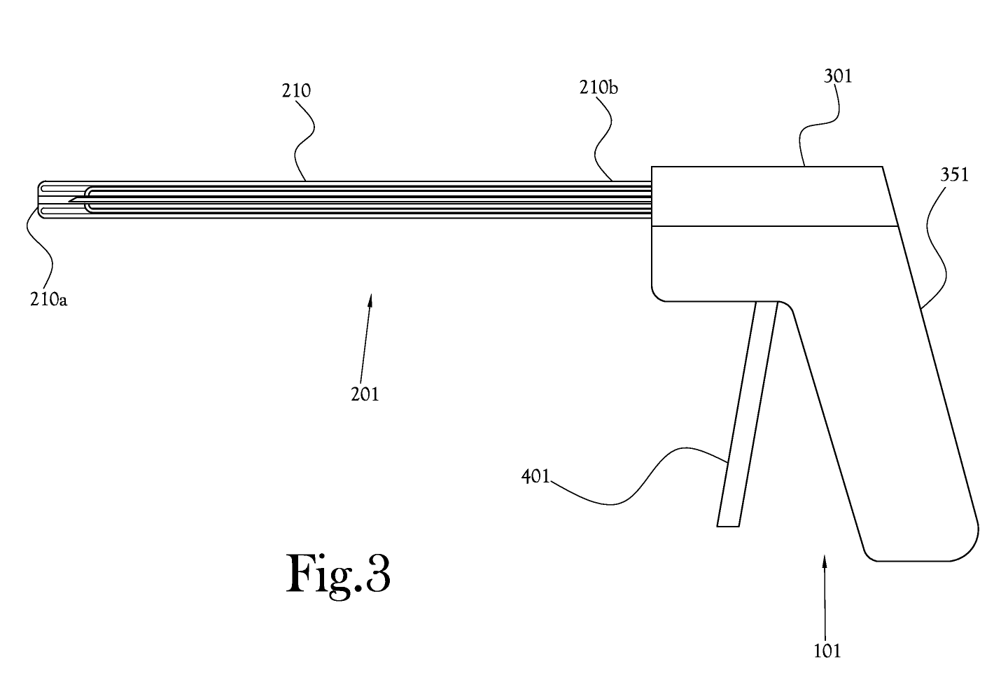

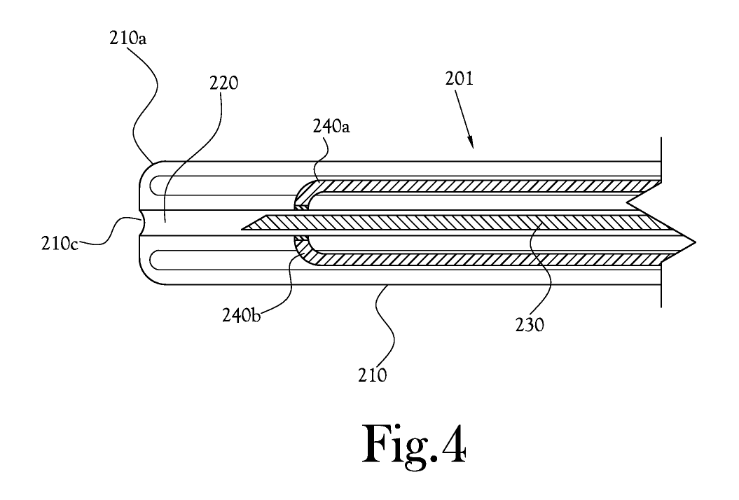

FIGS. 2 and 3 show one embodiment of the present invention. The analyzer 101 includes a probe 201, a housing 301 with a handle 351, a trigger 401, and a display 501 that shows the results of a pH test. The probe 201 includes a probe tube 210, which has a central longitudinal axis and two ends: a proximal end 210a, which defines an aperture 210c through which blood travels, and a distal end 210b, which is attached to the housing 301. As shown in FIG. 4, within the probe tube 210 is a capillary tube 220 running along the central longitudinal axis of the probe tube 210. Within the capillary tube 220 is a lancet 230 adapted to extend out the aperture in the proximal end 210a of the probe tube 210 during operation of the analyzer 101. Within the probe tube 210 are also two sensors 240a and 240b adapted to measure the pH of blood or other liquid in the capillary tube 220.

During use, an operator inserts the probe 201 into the birth canal until the proximal end 210a of the probe tube 210 has made contact with the scalp of the fetus. In some embodiments of the invention, the proximal end 210a of the probe tube 210 has a blunt, rounded surface, as shown in FIG. 4. The blunt proximal end 210a facilitates a positive placement of the probe 201 against the scalp. Next, the operator pulls the trigger 401 to advance the lancet 230. The lancet 230 makes a small incision in the scalp sufficient to produce blood. The trigger 401 is released, and the lancet 230 then retreats within the capillary tube 220. Blood flows into the capillary tube 220. In some embodiments, the probe tube 210 is fabricated from a substantially transparent material, such as clear plastic, thereby allowing the operator to visually confirm that the lancet 230 made a successful incision and that blood is flowing into the capillary tube 220. As blood fills the capillary tube 220, the sensors 240a and 240b measure the pH of the blood. The sensors 240a and 240b communicate their information to a controller within the housing 301; the controller in turn communicates with the display 501 to show the operator the pH of the drawn blood. In some embodiments, the display 501 is a digital display. In some embodiments, the display 501 comprises a number of lights (for example, LED lights) of different colors, with different colors representing a pH reading within a certain range (e.g. a green light representing a pH within a normal range, a yellow light representing a higher pH range, and a red light representing a dangerously high pH range).

The present invention provides a simplified apparatus for sampling fetal scalp blood by integrating lancing, blood sample collection, and blood analysis in one device. The blunt proximal end and clear probe tube facilitate quick and successful sample collection. Because the sensor is in close proximity to and in liquid communication with the capillary tube within which the blood sample is collected, the present invention also reduces the time between the beginning of the procedure and the delivery of blood pH analysis results. Because the lancet is positioned within the capillary tube, the probe tube has a smaller profile than other devices, allowing for its use on patients with smaller cervix dilation than required for other methods of fetal scalp blood analysis.

In some embodiments, the probe comprises a detachable unit that is disposed after one use. In some embodiments, the analyzer is adapted for use on adults to take real-time measurements of blood pH, as in emergency situations or in remote locations. In some embodiments, the analyzer described herein is modified, by changing sensors, to analyze other properties or characteristics of fetal scalp blood, such as lactose levels or a platelet count.

While the present invention has been illustrated by description of some embodiments, and while the illustrative embodiments have been described in detail, it is not the intention of the applicant to restrict or in any way limit the scope of the appended claims to such detail. Additional modifications will readily appear to those skilled in the art. The invention in its broader aspects is therefore not limited to the specific details, representative apparatus and methods, and illustrative examples shown and described. Accordingly, departures may be made from such details without departing from the spirit or scope of applicant's general inventive concept.

Having thus described the aforementioned invention, What is claimed is:

1. A fetal scalp blood analyzer comprising
a probe tube having a proximal end, a distal end, and central longitudinal axis, said probe tube being fabricated from a substantially transparent material, said probe tube defining an aperture at said proximal end of said probe tube,
a capillary tube adapted to collect a sample of blood, said capillary tube being positioned within said probe tube,
an extensible lancet adapted to produce an incision in the scalp for the purpose of drawing blood from the scalp, said lancet being positioned within said capillary tube, and
a sensor adapted to measure the pH of blood, said sensor being in liquid communication with the interior of said capillary tube.

2. The fetal scalp blood analyzer of claim 1 further comprising a display in communication with said sensor, said display being adapted to show pH as measured by said sensor.

3. The fetal scalp blood analyzer of claim 2 wherein said display is a digital display.

4. The fetal scalp blood analyzer of claim 1 wherein said proximal end of said probe tube defines a blunt, rounded surface adapted to facilitate secure placement of said probe tube against the scalp.

5. The fetal scalp blood analyzer of claim 4 further comprising a display in communication with said sensor, said display being adapted to show pH as measured by said sensor.

6. The fetal scalp blood analyzer of claim 5 wherein said display is a digital display.

7. A fetal scalp blood analyzer comprising
a capillary tube adapted to collect a sample of blood,
an extensible lancet adapted to produce an incision in the scalp for the purpose of drawing blood from the scalp, said lancet being positioned within said capillary tube, and
a sensor adapted to measure the pH of blood, said sensor being in liquid communication with said capillary tube.

8. The fetal scalp blood analyzer of claim 7 further comprising a display in communication with said sensor, said display being adapted to show pH as measured by said sensor.

9. The fetal scalp blood analyzer of claim 8 wherein said display is a digital display.

10. The fetal scalp blood analyzer of claim 8 wherein said display includes a series of colored lights.

11. The fetal scalp blood analyzer of claim 7 wherein said proximal end of said probe tube defines a blunt, rounded surface adapted to facilitate secure placement of said probe tube against the scalp.

12. The fetal scalp blood analyzer of claim 11 further comprising a display in communication with said sensor, said display being adapted to show pH as measured by said sensor.

13. The fetal scalp blood analyzer of claim 12 wherein said display is a digital display.

14. The fetal scalp blood analyzer of claim 12 wherein said display includes a series of colored lights.

13. A fetal scalp blood analyzer comprising
a probe tube having a proximal end, a distal end, and central longitudinal axis, said probe tube defining an aperture at the proximal end of said probe tube,
a capillary tube adapted to collect a sample of blood through said aperture, said capillary tube being positioned within said probe tube and extending along said central longitudinal axis of said probe tube,
an extensible lancet adapted to produce an incision in the scalp for the purpose of drawing blood from the scalp, said lancet being disposed within said capillary tube, and
a sensor adapted to measure a blood characteristic, said sensor being in liquid communication with said capillary tube.

14. The fetal scalp blood analyzer of claim 13 wherein said blood characteristic is the level of lactose in the blood.

15. The fetal scalp blood analyzer of claim 13 wherein said blood characteristic is the count of platelets in the blood.
